(19) United States
(12) Patent Application Publication
(10) Pub. No.: US 2024/0165426 A1
(43) Pub. Date: May 23, 2024

(54) COOLANT SUPPLY APPARATUS FOR ROTATING GANTRY, AND PARTICLE BEAM TREATMENT SYSTEM

(71) Applicants: KABUSHIKI KAISHA TOSHIBA, Toyko (JP); TOSHIBA ENERGY SYSTEMS & SOLUTIONS CORPORATION, Kawasaki-shi (JP)

(72) Inventors: Kazuhito TOMITA, Yokohama Kanagawa (JP); Yasuhiro YUGUCHI, Yokohama Kanagawa (JP); Shinichi TAKAMA, Yokohama Kanagawa (JP); Kiyohiko KITAGAWA, Yokohama Kanagawa (JP)

(73) Assignees: KABUSHIKI KAISHA TOSHIBA, Toyko (JP); TOSHIBA ENERGY SYSTEMS & SOLUTIONS CORPORATION, Kawasaki-shi (JP)

(21) Appl. No.: 18/419,816

(22) Filed: Jan. 23, 2024

Related U.S. Application Data
(63) Continuation of application No. PCT/JP2022/028700, filed on Jul. 26, 2022.

(30) Foreign Application Priority Data
Oct. 5, 2021 (JP) 2021-164005

(51) Int. Cl. A61N 5/10; A61N 5/00

(52) U.S. Cl. CPC A61N 5/1081; A61N 2005/002

(57) ABSTRACT

According to one embodiment, a coolant supply apparatus for a rotating gantry comprising: a rotating gantry that supports both an irradiation nozzle configured to radiate a particle beam and a transport unit configured to transport the particle beam to the irradiation nozzle and rotates around a horizontal axis directed in a horizontal direction; and at least one cable group that is configured by integrating a plurality of cables arranged in line along a band-shaped reinforcement member, is connected at one end to the rotating gantry, and is connected at another end to a stationary device.

### CROSS-REFERENCE TO RELATED APPLICATIONS

This application is a Continuation application of No. PCT/JP2022/028700, filed on Jul. 26, 2022, and the PCT application is based upon and claims the benefit of priority from Japanese Patent Application No. 2021-164005, filed on Oct. 5, 2021, the entire contents of which are incorporated herein by reference.

### TECHNICAL FIELD

Embodiments of the present invention relate to a coolant supply apparatus for a rotating gantry.

### BACKGROUND

When a treatment table of a rotating gantry is used in a particle beam treatment system, particle beams can be radiated in a state where a patient remains stationary, and thus, the burden on the patient can be reduced as compared with a case of using a fixed treatment table. However, the rotating gantry includes many devices inside, and these devices rotate together with the rotating gantry. Hence, the rotating gantry needs to be connected to stationary external devices by using many cables that are necessary for electric power, control, and communication. In the rotating gantry, cables are wound or unwound onto/from a spool each time the rotating gantry rotates.

Although the rotating gantry can be downsized by using superconducting electromagnets, hoses for supplying liquid helium for cooling down the superconducting electromagnets from the outside are needed. However, if a large number of cables are wound onto the spool, the cables are irregularly wound in some cases. In this context, when hollow flexible hoses are used for supplying the coolant to the superconducting electromagnets, the flexible hoses are irregularly wound so as to be twisted, resulting in interruption in supply of the coolant to the superconducting electromagnets. In one conventional technique, a cableveyor (registered trademark) is used to bundle the plurality of cables together. However, using the cableveyor increases not only the manufacturing cost but also the number of components in the apparatus, which complicates the control of rotating the rotating gantry.

### PRIOR ART DOCUMENT

### Patent Document

[Patent Document 1] JP H10-330037 A
[Patent Document 2] JP 2001-251748 A
[Patent Document 3] JP 2008-067908 A

### SUMMARY

### Problem to be Solved by Invention

An object of the present invention is to provide a coolant supply apparatus that is for a rotating gantry and can supply a coolant to a superconducting electromagnet without interruption.

### BRIEF DESCRIPTION OF DRAWINGS

FIG. 1 is a plan view illustrating an overall configuration of a particle beam treatment system according to the first embodiment.

FIG. 2 is a side view illustrating a rotating gantry.

FIG. 3 is a side view illustrating a spool of the rotating gantry.

FIG. 4 is a rear view of the rotating gantry corresponding to the cross-section taken along the line IV-IV of FIG. 3.

FIG. 5 is a rear view illustrating a cover of the spool.

FIG. 6 is a side view illustrating the brim disks and the cables according to the first embodiment.

FIG. 7 is a side view illustrating the brim disks and the cables according to the second embodiment.

FIG. 8 is a side view illustrating the brim disks and the cables according to the third embodiment.

FIG. 9 is a side view illustrating the brim disks and the cables according to the fourth embodiment.

FIG. 10 is a side view illustrating the brim disks and the cables according to the fifth embodiment.

### DETAILED DESCRIPTION

In one embodiment of the present invention, a coolant supply apparatus for a rotating gantry comprising: a rotating gantry that supports both an irradiation nozzle configured to radiate a particle beam and a transport unit configured to transport the particle beam to the irradiation nozzle and rotates around a horizontal axis directed in a horizontal direction; and at least one cable group that is configured by integrating a plurality of cables arranged in line along a band-shaped reinforcement member, is connected at one end to the rotating gantry, and is connected at another end to a stationary device.

According to embodiments of the present invention, it is possible to provide a coolant supply apparatus that is for a rotating gantry and can supply a coolant to a superconducting electromagnet without interruption.

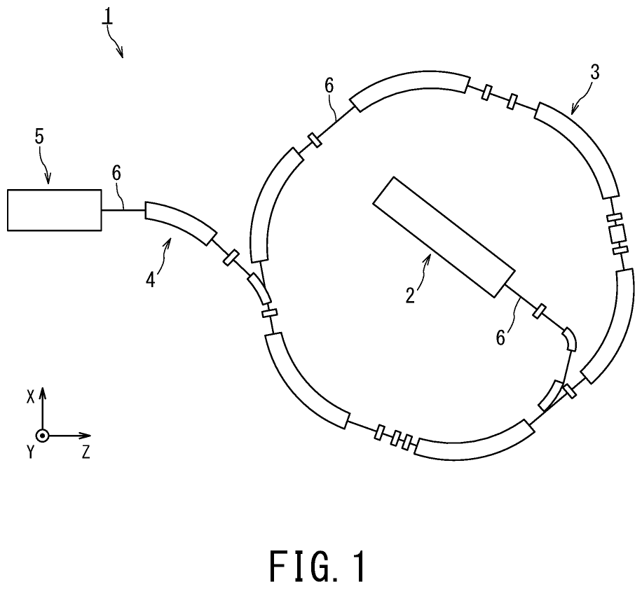

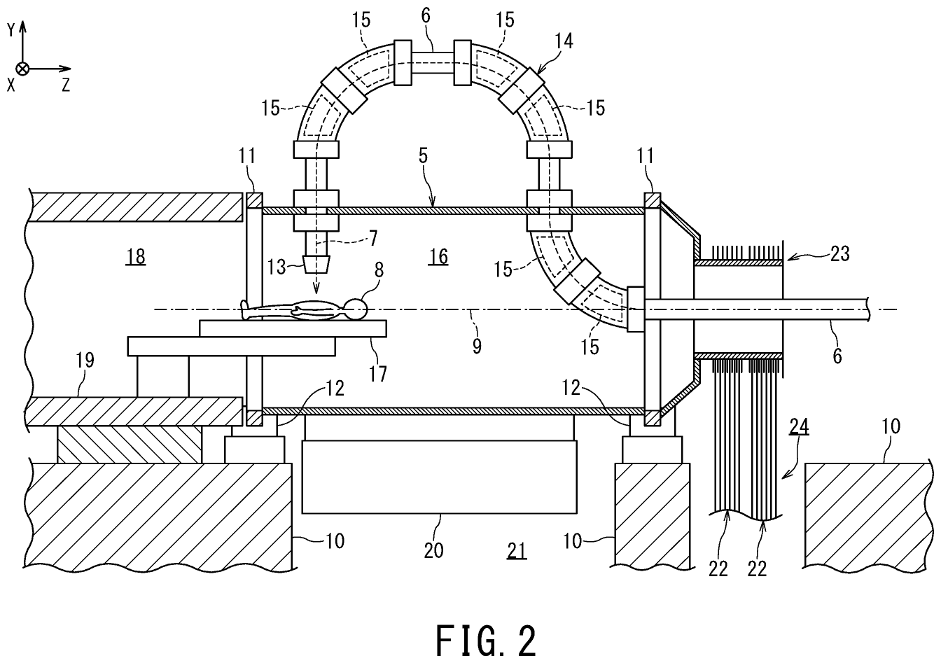

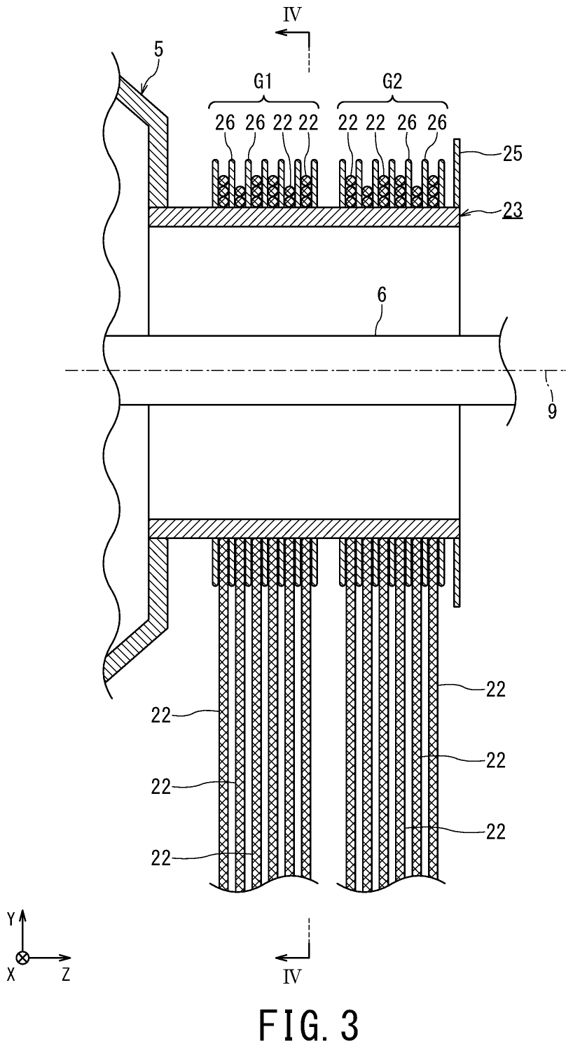

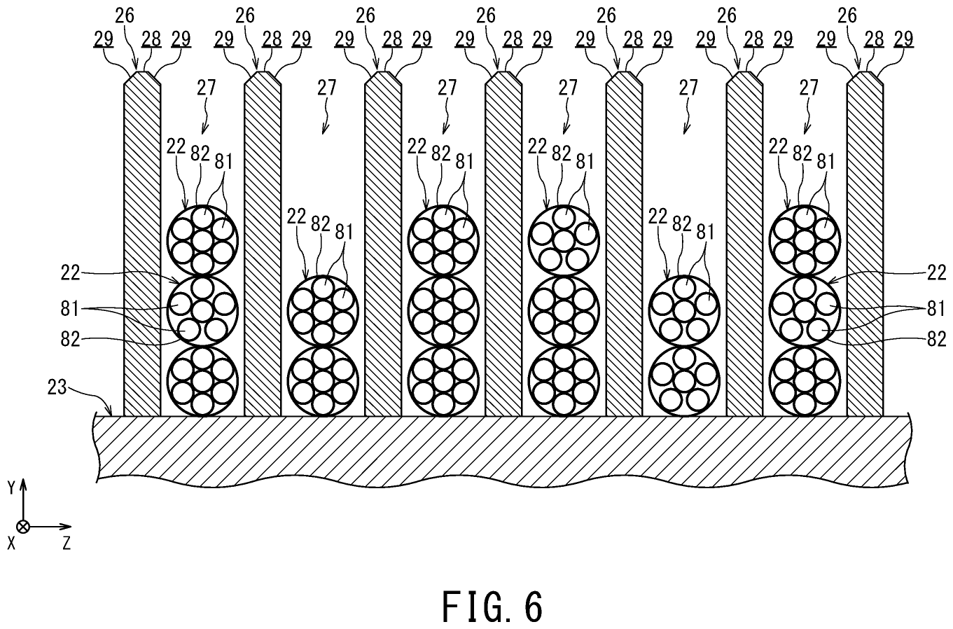

(First Embodiment) Hereinbelow, a description will be given of embodiments of a particle beam treatment system and a rotating gantry in detail by referring to the accompanying drawings. First, the first embodiment will be described by using FIG. 1 to FIG. 6. In the following description, the left side of the sheet of each of FIG. 2, FIG. 3, and FIG. 6 is assumed to be the front side of the rotating gantry, and the right side of the sheet of each of these figures is assumed to be the back side (i.e., the rear side) of the rotating gantry. In each figure, the axial direction of the rotating gantry is assumed to be the Z-axis direction in the orthogonal coordinate system, the vertical direction (i.e., the up-and-down direction) orthogonal to this Z-axis direction is assumed to be the Y-axis direction, and the horizontal direction orthogonal to both the Z-axis and the Y-axis is assumed to be the X-axis direction. Note that the X-axis direction and the Y-axis direction are sometimes referred to as the radial direction of the rotating gantry. Furthermore, the direction of rotating around the axis along the outer circumferential surface of the rotating gantry is sometimes referred to as the circumferential direction.

The reference sign 1 in FIG. 1 denotes the particle beam treatment system according to the first embodiment. In this particle beam treatment system 1, treatment is performed by irradiating a diseased tissue (cancer) of a patient as a target with particle beams such as carbon ions.

A radiation therapy technique with the use of the particle beam treatment system 1 is also referred to as a heavy ion beam cancer treatment technique. This technique is said to be able to damage a cancerous lesion (i.e., focus of disease) and minimize the damage to normal cells by pinpointing the cancerous lesion with carbon ions. Note that the particle beams are defined as radioactive rays heavier than electrons, and include proton beams and heavy ion beams, for example. Of these particle beams, heavy ion beams are defined as radioactive rays heavier than helium atoms.

As compared with the conventional cancer treatment using X-rays, gamma rays, or proton beams, the cancer treatment using heavy ion beams has characteristics that: (i) the ability to kill the cancerous lesion is higher; and (ii) the radiation dose is weak on the surface of the body of the patient so as to peak at the cancerous lesion. Thus, the number of irradiations and side effects can be reduced, and the treatment period can be shortened.

As shown in FIG. 1, the particle beam treatment system 1 includes a beam generator 2, a circular accelerator 3, a beam transport line 4, and a rotating gantry 5.

The beam generator 2 has an ion source of carbon ions, which are charged particles, and uses these carbon ions to generate a particle beam 7 (FIG. 2). The circular accelerator 3 has a ring shape in a plan view, and accelerates the particle beam 7 generated by the beam generator 2. The beam transport line 4 transports the particle beam 7 accelerated by the circular accelerator 3 to the rotating gantry 5. A patient 8 (FIG. 2) to be irradiated with the particle beam 7 is placed in the rotating gantry 5.

In this particle beam treatment system 1, first, the particle beam 7 of carbon ions generated by the beam generator 2 is inputted from the beam generator 2 to the circular accelerator 3. This particle beam 7 is accelerated to approximately 70% of the speed of light while orbiting the circular accelerator 3 approximately one million times. Thereafter, this particle beam 7 is guided to the rotating gantry 5 via the beam transport line 4.

The beam generator 2, the circular accelerator 3, and the beam transport line 4 are provided with vacuum ducts 6 (beam pipes), inside of which is vacuumized. The particle beam 7 passes the inside of the vacuum ducts 6. The vacuum ducts 6 of the beam generator 2, the circular accelerator 3, and the beam transport line 4 are integrated so as to form a transport path that guides the particle beam 7 to the rotating gantry 5. In other words, the vacuum ducts 6 are closed continuous space with a sufficient degree of vacuum to allow the particle beam 7 to pass through.

As shown in the cross-sectional view of FIG. 2, the rotating gantry 5 is an apparatus in a cylindrical shape. This rotating gantry 5 is installed in such a manner that the axis 9 of its cylindrical body is directed in the horizontal direction. The rotating gantry 5 can rotate around this horizontal axis 9.

The rotating gantry 5 is supported by a structure 10 of a building constituting a treatment facility in which the particle beam treatment system 1 is installed. For example, end rings 11 are fixed to the front portion and the rear portion of the main body of the rotating gantry 5. Below these end rings 11, rotary drivers 12 are provided. The rotary drivers 12 rotatably support the end rings 11 and include drive motors. These rotary drivers 12 are supported by the structure 10. The driving force of the rotary drivers 12 is applied to the rotating gantry 5 through the end rings 11, and thereby, the rotating gantry 5 is rotated around the horizontal axis 9.

The rotating gantry 5 is provided with the vacuum ducts 6 extending from the beam transport line 4 (FIG. 1). The vacuum ducts 6 are first guided from the rear side of the rotating gantry 5 into the inside along the horizontal axis 9. Further, the vacuum ducts 6 once extend outward from the outer circumferential surface of the rotating gantry 5, and then again extend toward the inside of the rotating gantry 5. The tip of the vacuum ducts 6 extends to a position close to the patient 8.

Of the vacuum ducts 6, the portion along the horizontal axis 9 of the rotating gantry 5 is provided with a predetermined rotation mechanism, which is not particularly illustrated. Of the vacuum ducts 6, the portion outside this rotating mechanism is stationary, and the portion inside this rotating mechanism rotates together with the rotation of the rotating gantry 5.

In addition, the rotating gantry 5 includes: an irradiation nozzle 13 configured to irradiate the patient 8 with the particle beam 7; and a transport unit 14 (or transport apparatus 14) configured to transport the particle beam 7 to the irradiation nozzle 13. In other words, the irradiation nozzle 13 and the transport unit 14 are supported by the rotating gantry 5.

Further, the transport unit 14 includes superconducting electromagnets 15 configured to generate a magnetic field that forms a path for transporting the particle beam 7. These superconducting electromagnets 15 are bending electromagnets configured to change the traveling direction of the particle beam 7 along the vacuum ducts 6 or quadrupole electromagnets configured to control convergence and divergence of the particle beam 7, for example.

The irradiation nozzle 13 is provided at the tip of the vacuum ducts 6 and radiates the particle beam 7 guided by the transport unit 14 toward the patient 8. The irradiation nozzle 13 is fixed to the inner circumferential surface of the rotating gantry 5. Note that the particle beam 7 is radiated from the irradiation nozzle 13 in the direction perpendicular to the horizontal axis 9.

Inside the rotating gantry 5, a treatment space 16 for performing particle beam therapy is provided. The patient 8 is placed on a treatment table 17 provided in this treatment space 16. This treatment table 17 can be moved with the patient 8 placed thereon. Positioning can be performed by moving this treatment table 17 in such a manner that the patient 8 on this treatment table 17 is moved to the irradiation position of the particle beam 7. Thus, the particle beam 7 can be radiated to an appropriate site such as the diseased tissue of the patient 8.

The patient 8 is placed at the position of the horizontal axis 9, and the irradiation nozzle 13 can be rotated around the stationary patient 8 by rotating the rotating gantry 5. For example, the irradiation nozzle 13 can be rotated around the patient 8 (i.e., around the horizontal axis 9) clockwise or counterclockwise in increments of 180° when viewed from the back. The particle beam 7 can be radiated from any direction around the patient 8. In other words, the rotating gantry 5 is an apparatus that can change the irradiation direction of the particle beam 7 guided by the beam transport line 4 with respect to the patient 8. Thus, the particle beam 7 can be radiated from the appropriate direction to the lesion site with higher precision while reducing the burden on the patient 8.

The particle beam 7 loses its kinetic energy at the time of passing through the body of the patient 8 so as to decrease its velocity and receive a resistance that is approximately inversely proportional to the square of the velocity, and stops rapidly when it decreases to a certain velocity. The stopping point of the particle beam 7 is referred to as the Bragg peak at which high energy is emitted. The particle beam treatment system 1 matches this Bragg peak with the position of the lesion tissue (i.e., affected part) of the patient 8, and thus, can kill only the lesion tissue while suppressing the damage to normal tissues.

The treatment space 16 provided inside the rotating gantry 5 is formed so as to be integrated with a treatment room 18 that is located on the front side of the rotating gantry 5. Note that the treatment table 17 is fixed to a floor 19 of the stationary treatment room 18. In other words, it is configured in such a manner that the position of the treatment table 17 does not change regardless of the rotation of the rotating gantry 5 and the irradiation nozzle 13.

Of the outer circumferential surface of the rotating gantry 5, on the opposite side of the portion where the transport unit 14 is provided, a counterweight 20 is fixed. This counterweight 20 is provided in order to maintain balance with the transport unit 14 around the rotating gantry 5. In other words, the weight of the counterweight 20 is set so as to correspond to the weight of the transport unit 14. In addition, a weight pit 21 formed in a concave shape in the structure 10 is provided below the rotating gantry 5 in such a manner that the counterweight 20 can pass through the weight pit 21 along with the rotation of the rotating gantry 5.

Further, a plurality of cables 22 are led from the outside to the rotating gantry 5. These cables 22 include power supply cables, signal lines, and flexible coolant hoses, for example. These cables 22 are provided in order to supply electric power and transmit control signals to specific devices installed in the rotating gantry 5. These cables 22 include flexible hoses 81 (FIG. 6) that supply a coolant to the superconducting electromagnets 15 included in the transport unit 14.

At the rear of the rotating gantry 5, a spool 23 is provided. The spool 23 winds or unwinds the cables 22 along with the rotation of the rotating gantry 5. The axis of the spool 23 coincides with the horizontal axis 9 of the rotating gantry 5.

Below the spool 23, a cable pit 24 formed in a concave shape in the structure 10 is provided. In the cable pit 24, the cables 22 hanging down from the spool 23 can be disposed. The width dimension of the cable pit 24 in the X-axis direction is set to be larger than the diameter of the spool 23.

As shown in the cross-sectional view of FIG. 3, the spool 23 is provided so as to protrude rearward from the rear portion of the rotating gantry 5. This spool 23 is a cylindrical portion, and is formed to have a smaller diameter than the diameter of the main body of the rotating gantry 5. This spool 23 includes one disk-shaped flange 25, a plurality of disk-shaped brim disks 26, and a plurality of concave lanes 27 (FIG. 6) that hold the cables 22.

The flange 25 is provided at the rear end of the spool 23. The plurality of brim disks 26 are arranged side by side in the axial direction (i.e., in the Z-axis direction) between the flange 25 and the rotating gantry 5. These brim disks 26 are formed to have a smaller diameter than the diameter of the flange 25. In addition, the rear brim disks 26, which are closest to the flange 25, are located at a distance from the flange 25. The plurality of lanes 27 (FIG. 6) are formed between the respective brim disks 26.

As shown in the cross-sectional view of FIG. 6, each lane 27 accommodates a plurality of cables 22. For example, one lane 27 accommodates two or three cables 22. Note that the number of cables 22 to be accommodated per one lane 27 may be four or more.

When the cables 22 are wound in the circumferential direction of the spool 23, the plurality of cables 22 are arranged in line in the axial direction (i.e., in the Z-axis direction) of the spool 23 and in line in the radial direction (i.e., in the X-axis direction and the Y-axis direction) of the spool 23 in accordance with the arrangement of the brim disks 26 and the lanes 27. In addition, when the plurality of cables 22 hang down from the spool 23, the cables 22 are arranged in line in the axial direction (i.e., in the Z-axis direction) of the spool 23 and in line in the horizontal direction (i.e., in the X-axis direction).

Note that the width of each lane 27 may be different depending on the number of the cables 22 to be accommodated or thickness of each cable 22. In addition, a plurality of cables 22 of different types or different thicknesses may be accommodated in one lane 27.

On the circumferential surface 28 of each brim disk 26, both corners are cut out to form chamfered portions 29 (i.e., bevels 29). In other words, the chamfered portions 29 are formed around the periphery of the brim disks 26. In this configuration, when the cables 22 are accommodated in the lanes 27, the cables 22 are less likely to be caught on the brim disks 26, thereby, the friction or tension on the cables 22 being caught on the brim disks 26 can be reduced, and consequently, the cables 22 are prevented from being irregularly wound.

For example, the chamfered portions 29 are inclined surfaces that are inclined at approximately 45° with respect to the protruding direction of the brim disks 26. Since these chamfered portions 29 are provided, the inlet width of each lane 27 is widened so as to allow the cables 22 to be smoothly accommodated in the lanes 27.

Even when the chamfered portions 29 are provided, part of the circumferential surface 28 of each brim disk 26 remains. For example, the circumferential surface 28 of the tip of each brim disk 26 remains. This structure can prevent the cables 22 from being cut or being worn out even if the cables 22 are get caught on the brim disk 26.

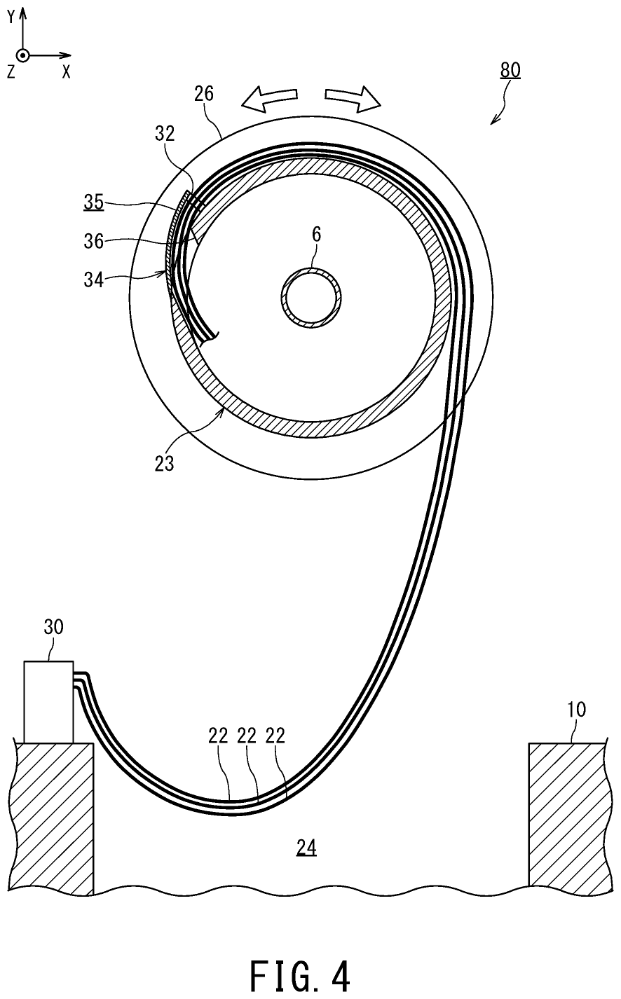

As shown in FIG. 4, each cable 22 is connected at one end to the spool 23 of the rotating gantry 5, and is connected at the opposite end to a stationary fixing device 30. The fixing device 30 is fixed to the structure 10, for example. The plurality of cables 22 are composed of power lines for supplying electric power, signal lines for transmitting control signals, and flexible hoses 81 (FIG. 6) for supplying the coolant, for example. The fixing device 30 is composed of a power supply, a terminal block, and a coolant supply pump, for example. Although FIG. 4 is a rear view of the rotating gantry 5, for the sake of facilitating understanding, respective illustrations of the main body of the rotating gantry 5, the rotary drivers 12, and the transport unit 14 are omitted in FIG. 4.

The particle beam treatment system 1 according to the present embodiment is provided with a coolant supply apparatus 80 for the rotating gantry 5. This coolant supply apparatus 80 includes at least the cables 22. The cables 22 of the coolant supply apparatus 80 are provided in order to supply the coolant via the spool 23 to the superconducting electromagnets 15 of the transport unit 14 provided in the rotating gantry 5.

As shown in the cross-sectional view of FIG. 6, each cable 22 is formed by bundling a plurality of flexible hoses 81 so as to form a circular shape in a cross-sectional view. For example, five or six flexible hoses 81 are bundled, and a protective tape 82 is spirally wrapped around the outer circumference of this bundle in such a manner that these flexible hoses 81 and the protective tape 82 covering the surface form one cable 22. In addition, a plurality of flexible hoses 81 may be bundled and accommodated in one large-diameter tube (not shown) so as to form one cable 22.

The flexible hoses 81 are hollow inside (FIG. 6) and are provided in order to supply the coolant such as liquid helium and liquid nitrogen to the superconducting electromagnets 15 (FIG. 2). Each flexible hose 81 is configured as a pressure-resistant hose in which metal wires are woven to increase its pressure resistance, and can supply the coolant at a predetermined pressure.

In addition, the plurality of flexible hoses 81 to be bundled together as one cable 22 are the same as each other in type, thickness, and hardness (rigidity). In this configuration, the flexible hoses 81 bundled as one cable 22 can be bent to the same degree, which makes it easier to wind this cable 22 onto the spool 23. Note that the plurality of flexible hoses 81 to be bundled together as one cable 22 may be different from each other in terms of type, thicknesses, and rigidity.

As shown in FIG. 3 and FIG. 4, the plurality of cables 22 are divided or classified into the first group G1 and the second group G2. This division or classification into the first group G1 and the second group G2 may be performed by type of the cables 22 or by the device to which the cables 22 are connected. In accordance with this division, a plurality of brim disks 26 around which the plurality of cables 22 of the first group G1 are wound are provided, and a plurality of brim disks 26 around which the plurality of cables 22 of the second group G2 are wound are provided.

The cables 22 of the first group G1 are different from the cables 22 of the second group G2 in the direction of being wound around spool 23. For example, when the rotating gantry 5 rotates counterclockwise in a rear view, the cables 22 of the first group G1 are wound onto the spool 23, whereas the cables 22 of the second group G2 are unwound from the spool 23. Conversely, when the rotating gantry 5 rotates clockwise, the cables 22 of the first group G1 are unwound from the spool 23, whereas the cables 22 of the second group G2 are wound onto the spool 23.

In FIG. 4, for the sake of facilitating understanding, only the cables 22 of the first group G1 are illustrated and the cables 22 of the second group G2 are omitted. In the actual rear view, the cables 22 of the first group G1 and the cables 22 of the second group G2 hanging down from the spool 23 appear to intersect each other at the cable pit 24.

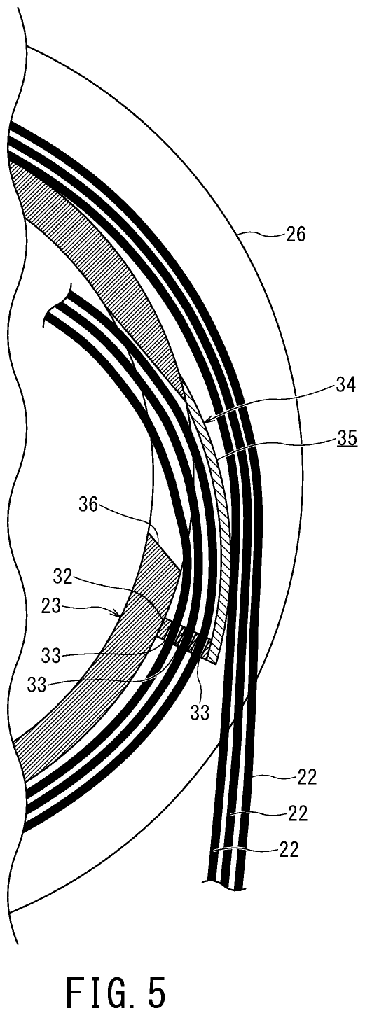

As shown in FIG. 4 and FIG. 5, the coolant supply apparatus 80 further includes connector portions 32, penetration portions 33, and a cover 34. Note that the configuration of the coolant supply apparatus 80 may include all or any one of the rotating gantry 5, the spool 23, and the brim disks 26.

The connector portions 32 are provided so as to correspond to the lanes 27 (FIG. 6) that hold the cables 22 in the spool 23, and protrude in the radial direction of the spool 23. For example, one connector portion 32 is provided so as to correspond to the plurality of lanes 27 of the first group G1. The connector portions 32 are, for example, plate members or blocks that are provided so as to protrude in the radial direction from the outer circumferential surface of the spool 23.

The penetration portions 33 are through holes formed in each of the connector portions 32, and are portions that penetrate the connector portions 32 in the circumferential direction and pass the cables 22 from the outside to the inside of the spool 23. Of the spool 23, the portion corresponding to the penetration portions 33 is provided with a penetration window 36. The cables 22 are introduced into the rotating gantry 5 through the penetration portions 33 and the penetration window 36. Further, the cables 22 are connected to devices such as the superconducting electromagnets 15 (FIG. 2) provided in the rotating gantry 5. Note that cables 22 are fixed to the positions of the penetration portions 33. Each cable 22 is wound circumferentially from the fixed penetration portions 33 along the outer circumference of the spool 23 (lanes 27).

The cover 34 extends from the lanes 27 to the tips of the connector portions 32. This cover 34 is a member having an inclined surface 35 that is inclined with respect to the outer circumferential surface of the spool 23. The portion around the penetration window 36 of the spool 23 is covered with this cover 34. In this configuration, the cables 22 can be wound gently from the lanes 27 to the tips of connector portions 32, and thus, buckling of the cables 22 can be prevented. The above-described “buckling of the cables 22” means that the cables 22 are bent significantly to the extent that the inside of the cables 22 is crushed or the function of the cables 22 is impaired.

In the first embodiment, the cables 22 are not bent significantly at the portion around the penetration portions 33, which can prevent reduction in ability to supply the coolant to the superconducting electromagnets 15.

Since the cables 22 are fixed by using the connector portions 32, even if one cable 22 is partially twisted inside the spool 23, this twisting is not transmitted to the other portion of this cable 22 outside the spool 23. Thus, twisting of the cables 22 to be wound onto the spool 23 can be prevented.

When the connector portions 32 are provided, at the time of replacing the cables 22, this replacement maintenance can be performed separately for the inside of the spool 23 (i.e., inside of the connector portions 32) and for the outside of the spool 23 (i.e., outside of the connector portions 32). For example, a connection portion is provided at the region around the connector portions 32 in such a manner that one cable 22 can be attached and detached at this connection portion, and maintenance of this one cable 22 is performed separately for the inside of the spool 23 and for the outside of the spool 23. In this configuration, the burden of the maintenance work can be reduced.

Since the plurality of flexible hoses 81 are bundled into one cable 22 in the first embodiment, the respective flexible hoses 81 are prevented from being twisted due to irregular winding, which can achieve smooth supply of the coolant to the superconducting electromagnets 15 without interruption.

Although the plurality of flexible hoses 81 are bundled together to form one cable 22 in the first embodiment, another aspect may be adopted. For example, a plurality of power lines or a plurality of signal lines may be bundled together to form one cable 22. In addition, the flexible hoses 81, the power lines, and the signal lines may be bundled together to form one cable 22.

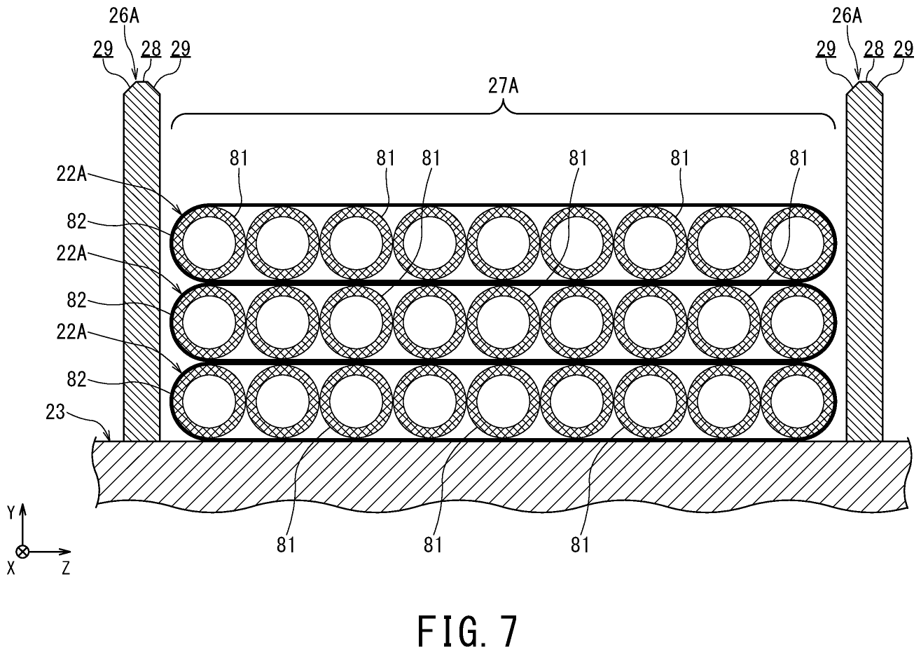

(Second Embodiment) Next, the second embodiment will be described by using FIG. 7. The same components as those shown in the above-described embodiments are denoted by the same reference signs, and duplicate descriptions are omitted.

Each cable 22A of the second embodiment is in a band shape (i.e., a flat plate shape) formed by integrating a plurality of flexible hoses 81 in parallel with each other. In other words, one cable 22A is formed by flattening and integrating the plurality of flexible hoses 81 to form an oval shape in a cross-sectional view.

For example, nine flexible hoses 81 are aligned in a straight line in a cross-sectional view, and the outer peripheries of the aligned flexible hoses 81 are spirally wrapped with the protective tape 82 in such a manner that these flexible hoses 81 and this protective tape 82 covering the surface form one cable 22 as a whole. In addition, the plurality of flexible hoses 81 may be accommodated in one tube (not shown) in the state of being aligned so as to form one cable 22.

The spool 23 is provided with at least two brim disks 26A. Between these brim disks 26A, a concave lane 27A having an inlet dimension larger than the width dimension of each cable 22 is formed.

Note that the plurality of cables 22A are held for one lane 27A in the state of being stacked in the radial direction of the spool 23. In other words, the spool 23 can wind or unwind the plurality of cables 22A in the state where these cables 22A are stacked in the radial direction. In this configuration, the plurality of cables 22A can be orderly wound onto the spool 23 without causing irregular winding.

Since the cables 22A are band-shaped in the second embodiment, the cables 22A are unlikely to be irregularly wound, thereby, each flexible hose 81 is prevented from being twisted, which achieves smooth supply of the coolant to the superconducting electromagnets 15 (FIG. 2) without interruption. In addition, the installation number of the brim disks 26A can be reduced by flattening and integrating the cables 22A.

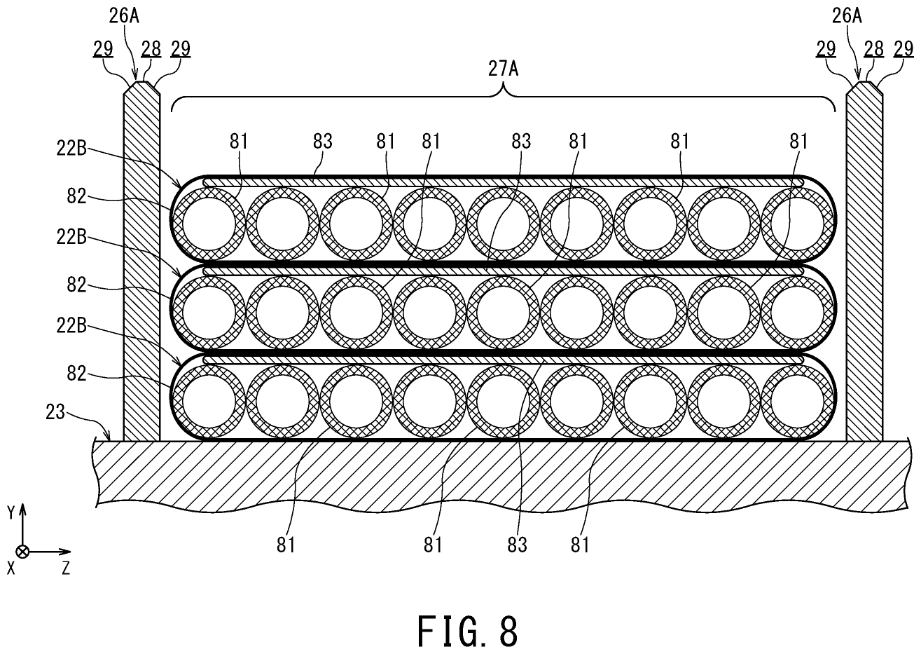

(Third Embodiment) Next, the third embodiment will be described by using FIG. 8. The same components as those shown in the above-described embodiments are denoted by the same reference signs, and duplicate descriptions are omitted.

Each cable 22B of the third embodiment is in a band shape (i.e., a flat plate shape) formed by integrating the plurality of flexible hoses 81 in parallel with each other. Furthermore, each cable 22B includes a band-shaped reinforcement member 83.

For example, nine flexible hoses 81 are aligned along one reinforcement member 83, and the outer peripheries of the aligned flexible hoses 81 and the reinforcement member 83 are spirally wrapped with the protective tape 82. In other words, the plurality of flexible hoses 81 are integrated together with the reinforcement member 83 by using the protective tape 82. Further, these flexible hoses 81, the reinforcement member 83, and the protective tape 82 form one cable 22B as a whole.

The reinforcement member 83 is provided at the portion that is the outer peripheral side of each band-shaped cable 22B when the cables 22B are wound around the spool 23. This configuration makes it easier to stack the cables 22B in the radial direction of the spool 23.

In the third embodiment, the reinforcement member 83 makes each cable 22B insusceptible to twisting, and thus, twisting of the flexible hoses 81 can be suppressed. Further, in manufacture of the cables 22B, it becomes easier to arrange the flexible hoses 81 in a straight line in a cross-sectional view, which facilitates manufacture of the cables 22B.

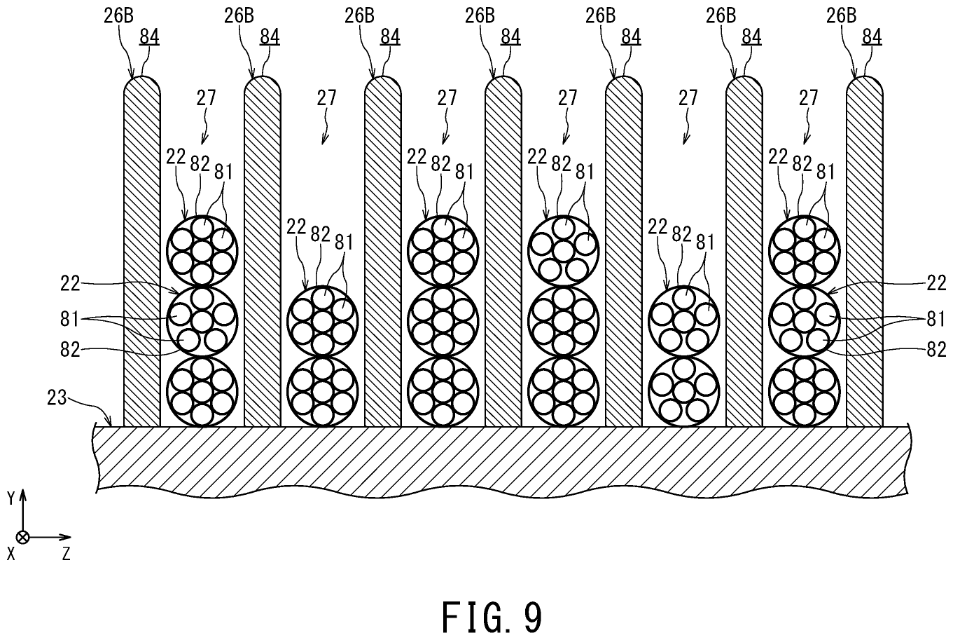

(Fourth Embodiment) Next, the fourth embodiment will be described by using FIG. 9. The same components as those shown in the above-described embodiments are denoted by the same reference signs, and duplicate descriptions are omitted.

Each brim disk 26B of the fourth embodiment has a semicircular periphery in a cross-sectional view. In other words, the circumferential surface of each brim disk 26B forms a curved surface 84. This curved surface 84 constitutes a chamfered portion of the fourth embodiment. In addition, on the circumferential surface of each of the brim disk 26B, both corners may be curved to form so-called round chamfering portions.

Since the circumferential surface of each brim disk 26B is the curved surface 84 in the fourth embodiment, each cable 22 is less likely to be caught in the brim disk 26B when being accommodated in the lane 27, thereby, the friction or tension on the cables 22 being caught on the brim disks 26 can be reduced, and consequently, the cables 22 are prevented from being irregularly wound. Even if the cable 22 is caught on the brim disk 26, the above-described configuration prevents the cable 22 from being cut or being worn out.

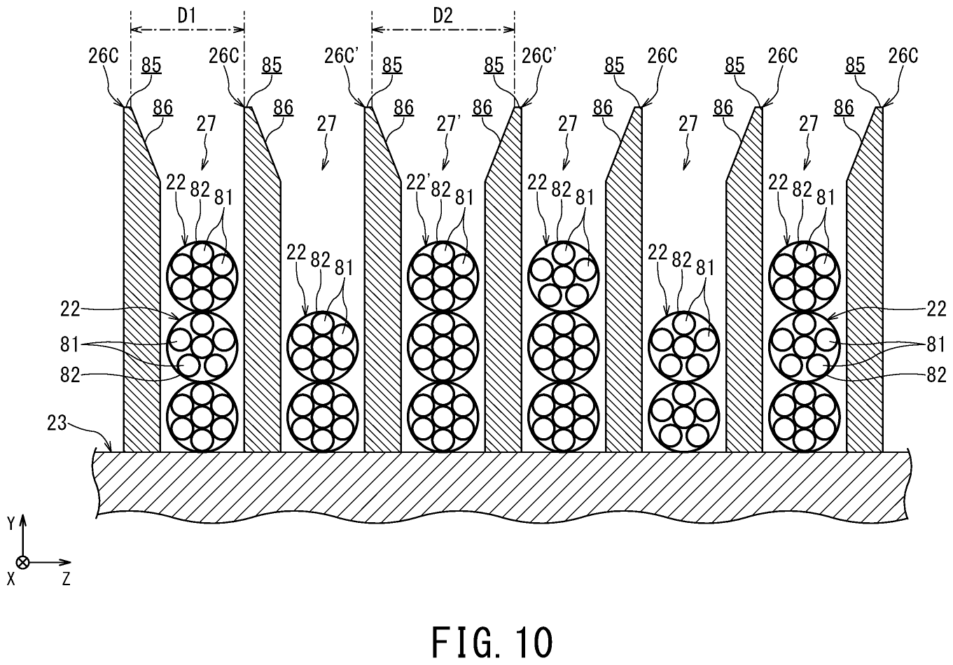

(Fifth Embodiment) Next, the fifth embodiment will be described by using FIG. 10. The same components as those shown in the above-described embodiments are denoted by the same reference signs, and duplicate descriptions are omitted.

On the peripheral surface 85 of each brim disk 26C in the fifth embodiment, a chamfered portion 86 is formed by cutting out one corner. In other words, the chamfered portion 86 is formed on only one side of each brim disk 26C. For example, the chamfered portion 86 is an inclined surface having an inclination of about 30° with respect to the protruding direction of the brim disk 26C.

Of the two brim disks 26C arranged on both sides of one lane 27, one brim disks 26C is disposed so as to direct its surface formed as the chamfered portion 86 to the lane 27, and the other brim disks 26C is disposed so as to direct the surface without being formed as the chamfered portion 86 to the lane 27. In this configuration, the inlet dimension D1 of each lane 27 can be made wider than at least the case where the chamfered portion 86 is not formed.

For a specific lane 27′, both brim disks 26C′ on both sides of this lane 27′ are arranged in such a manner that both surfaces formed as the chamfered portions 86 face this lane 27′. In this configuration, the inlet dimension D2 can be at least wider than the inlet dimension D1 of the other lanes 27. For example, when the lane 27′ that is more difficult for the cable 22′ to enter than the other lanes 27 can be specified in advance, the chamfered portions 86 are arranged on both sides of this lane 27′ so as to create a wide inlet dimension D2. This configuration makes the cable 22′ easier to enter the lane 27′, and thus, the cables 22 are prevented from being irregularly wound.

As above, although the coolant supply apparatus for a rotating gantry have been described on the basis of the first to fifth embodiments, the configuration applied in any one of the embodiments may be applied to other embodiments or the configurations in the respective embodiments may be applied in combination.

For example, when the band-shaped cables 22A and 22B of the second or the third embodiment are held by the brim disks 26C of the fifth embodiment, the brim disks 26C are provided in such a manner that the chamfered portions 86 of both brim disks 26C sandwiching the cables 22A and 22B are directed toward these cables 22A and 22B and thereby the inlet width is widened.

Although a facility configured to perform heavy-ion-beam cancer treatment is exemplified in the above-described embodiments, the above-described embodiments can also be applied to other facilities. For example, the above-described embodiments may be applied to a facility that performs proton-beam cancer treatment.

According to at least one embodiment described above, buckling of the cables for suppling the coolant to the superconducting electromagnets can be prevented by providing the penetration portion that penetrates the spool in the circumferential direction of the spool and causes the cables to pass from the outside to the inside of the spool.

Moreover, smooth supply of the coolant to the superconducting electromagnets of the transport unit can be achieved without any interruption by forming each cable into the band-shaped cable in which the plurality of flexible hoses for supplying the coolant to the superconducting electromagnets are arranged in parallel with each other and integrated.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A coolant supply apparatus for a rotating gantry comprising:
a rotating gantry that supports both an irradiation nozzle configured to radiate a particle beam and a transport unit configured to transport the particle beam to the irradiation nozzle and rotates around a horizontal axis directed in a horizontal direction; and
at least one cable group that is configured by integrating a plurality of cables arranged in line along a band-shaped reinforcement member, is connected at one end to the rotating gantry, and is connected at another end to a stationary device.

2. The coolant supply apparatus for the rotating gantry according to claim 1, further comprising a spool, wherein:
the at least one cable group comprises a plurality of cable groups; and
the spool is provided on the rotating gantry and winds or unwinds the plurality of cable groups in a radially stacked state.

3. The coolant supply apparatus for the rotating gantry according to claim 2, further comprising:
a connector portion that is provided corresponding to a lane configured to hold the plurality of cable groups in the spool and protrudes in a radial direction of the spool; and
a plurality of penetration portions that are formed in the connector portion, penetrate connector portion in a circumferential direction of the spool, and pass the plurality of cable groups from outside to inside of the spool.

4. The coolant supply apparatus for the rotating gantry according to claim 3, further comprising a cover that has an inclined surface extending from the lane to a tip of the connector portion.

5. The coolant supply apparatus for the rotating gantry according to claim 2, wherein the spool includes at least one disc-shaped brim disk having a chamfered portion that is formed on a periphery.

6. A particle beam treatment system comprising:
the coolant supply apparatus for a rotating gantry according to claim 1;
a treatment table configured to perform positioning by moving a patient to an irradiation position of the particle beam guided by the transport unit in a direction perpendicular to the horizontal axis;
a beam generator configured to generate the particle beam; and
an accelerator configured to accelerate the particle beam.
